(19) United States
(12) Patent Application Publication
(10) Pub. No.: US 2016/0195518 A1
(43) Pub. Date: Jul. 7, 2016

(54) EXPERIMENTAL METHODS FOR CONDUCTING COMPETITIVE BINDING ASSAYS

(71) Applicant: Sapidyne Instruments Inc., Boise, ID (US)

(72) Inventors: Thomas R. Glass, Boise, ID (US); Steve J. Lackie, Boise, ID (US)

(21) Appl. No.: 14/591,624

(22) Filed: Jan. 7, 2015

(51) Int. Cl. G01N 33/53

(52) U.S. Cl. CPC G01N 33/5306

(57) ABSTRACT

The present disclosure is directed toward improved methods of conducting a competitive binding assay or experiment. The methodology includes utilizing either a positive control, a negative control, or both in order to scale experimentation results to results that can be utilized to obtain a precise measurement of the kinetic rate constant (the off rate denoted as k_{off}) describing the dissociation of a non-covalent complex such as an antibody antigen complex, receptor ligand complex, etc.

### BACKGROUND AND SUMMARY OF THE INVENTION

The purpose of the Summary of the Invention is to enable the public, and especially the scientists, engineers, and practitioners in the art who are not familiar with patent or legal terms or phraseology, to determine quickly from a cursory inspection, the nature and essence of the technical disclosure of the application. The Summary of the Invention is neither intended to define the invention of the application, which is measured by the claims, nor is it intended to be limiting as to the scope of the invention in any way.

The goal of the process described herein is to obtain a precise measurement of the kinetic rate constant (the off rate denoted as k_{off}) describing the dissociation of a non-covalent complex such as an antibody antigen complex, receptor ligand complex, etc. The overall rate at which a non-covalent complex dissociates into its constituent molecules is given by the product of k_{off} times the concentration of the non-covalent complex, or,

Dissociation rate=k_{off}*[AB]

where [AB] represents the concentration of the non-covalent complex of generic constituent molecules A and B. The method for determining k_{off} relies on introducing a third molecule C which is distinguishable from B and binds competitively to A. Molecule B and C do not necessarily have to bind to the same epitope on A but their binding must be mutually exclusive so that molecule A can bind either molecule B or molecule C but cannot bind both simultaneously.

Various means to distinguish molecule B from molecule C are envisioned, including a covalent label (fluorescent, radioactive, enzymatic, colorimetric, electrochemical etc.) or a structural difference, either naturally occurring (a closely related but detectably different cross reacting molecule, or, a completely unrelated molecule that happens to bind in a mutually exclusive manner) or engineered into molecule B or C (his tag, flag tag, biotinylated, etc.) or the like. Detection of the distinguishable molecule may occur on any of numerous platforms including mass spectrometry, surface plasmon resonance (SPR) based biosensor, enzyme-linked immunosorbent assay (ELISA), flow cytometry, quartz crystal microbalance, micro calorimetry, various immunoassay platforms, and various other biosensors.

While the competitive binding analysis is thought to be applicable to short time frames, researchers and scientists have had high difficulty in applying the process at long time frames. This is shown, for example, in the reference Dowling, M. R. et al, Quantifying the association and dissociation rates of unlabelled antagonists at the muscarinic M3 receptor. British Journal of Pharmacology. 148:927-937 (2006). The publication specifically discusses the difficulty and likely inapplicability of the competitive binding analysis at long time durations.

In a preferred embodiment of the invention molecule C is produced from molecule B by covalent attachment of a fluorescent label and the detection platform the KinExA instrument. For a specific example, consider the case in which molecule C is a fluorescently labeled version of molecule B. The off rate measurement then consists of measuring the concentration of C, either free (not complexed with A) or bound (AC complex) in solution as a function of time. The measurement starts with a substantially large fraction of both the total A and the total C existing in the complexed form and concentration of AC complex is either measured directly (AC complex measured) or indirectly (free C measured). In general, the off rate for molecules A and C is often of less interest than the off rate of A and B, because C will often be, as in this example, a modified version of B. The preferred embodiment overcomes this in the following manner. Solutions of AB complex (in the absence of C) and AC complex (in the absence of B) are prepared. Generally concentrations are chosen such that once equilibrium is achieved essentially all of the molecule B in the AB complex solution exists in the complexed form and essentially all of the molecule C in the AC complex solution exists in the complexed form. Once the equilibrium of the two solutions is achieved, molecule C is introduced to the AB complex solution and molecule B is introduced to the AC complex solution. Both of these solutions then proceed to a new competitive equilibrium in which both solutions have a balance of AB and AC along with complementary balances of B and C. In the preferred embodiment, the total quantities of A, B, and C are identical in both mixtures and thus both mixtures move to the same equilibrium from different starting points. The rate at which the solution that begins primarily as a mixture of AB and C moves to a new equilibrium with a mixture of AB, AC, B, and C is strongly influenced by the k_{off} of the AB complex while the rate at which the solution that begins primarily as a mixture of AC and B moves to its new equilibrium with, in the preferred embodiment the same equilibrium, is strongly influenced by the k_{off} of the AC complex.

Because the final equilibrium is competitive with molecules B and C competing for a limited amount of molecule A, the rates at which the two mixtures move to the final equilibrium also depend on the on rates (association rate constant, kₒₙ) of molecules B and C for A and on the concentrations of A, B, and C. The dependency arises because when a molecule of AB, for example, dissociates into A and B, molecule A may then bind to either B or C or it may persist unbound in solution. The relative likelihood of these three possibilities depends on the relative concentrations of A, B, and C and on the on rates of A-B and A-C. For this reason, the preferred embodiment of the invention includes a step of accurately measuring the relative concentrations of the reactants and of measuring at least one of the two on rates.

It is anticipated that in many cases the present invention will be applied in cases where the off rates are very slow and subsequently the time for the mixtures to reach the final equilibrium will be measured days, weeks, or months. Under these circumstances it is critical to provide a reference to monitor the response of the measurement system and the viability of the reactants. In the preferred embodiment two references are included, one monitoring the maximum signal (either AC or C depending on whether the complex or free C is measured) and another monitoring the minimum signal. It is anticipated that various different solutions may be used for the references. For example, the minimum signal reference mentioned may represent the non specific binding of the labeled C and therefore be free of A and B but in some configurations it may be desirable to capture C (for example via a biotin tag) and then label A (for example using a fluorescently labelled anti A antibody) in which case the NSB control may include A and B but not C. Other possibilities also exist; the exact reference solutions used will depend on the results of preliminary investigations into the source of the baseline signal (usually the nsb) on the specific system and measurement format selected. The purpose of the reference solutions remains constant however and is to provide a scaling reference (to correct instrument drift for example) and an offset reference (to correct changing NSB for example).

In addition, it is desirable to avoid the possibility of pipetting error affecting the relative concentrations in the various solutions so the preferred embodiment includes preparing larger volumes of A, B, and C and then splitting and combining these to form first the mixtures of AB and AC and then the mixtures of AB+C and AC+B. These preparations and mixtures will include the control solutions. Examples of sample preparation for the cases of measuring AC and measuring C are diagrammed in FIGS. 10 and 11 respectively.

Still other features and advantages of the claimed invention will become readily apparent to those skilled in this art from the following detailed description describing preferred embodiments of the invention, simply by way of illustration of the best mode contemplated by carrying out my invention. As will be realized, the invention is capable of modification in various obvious respects all without departing from the invention. Accordingly, the descriptions of the preferred embodiments are to be regarded as illustrative in nature, and not as restrictive in nature.

### BRIEF DESCRIPTION OF THE DRAWINGS

FIG. 1 illustrates a background concept of a labeled partner and unlabeled partner competing for binding with a limited partner.

FIG. 2 illustrates an overview of an example competitive binding assay detailing molecule interaction in an experiment measuring the percent free labeled partner at the beginning of the time illustrated on the graph.

FIG. 3 illustrates an overview of an example competitive binding assay detailing molecule interaction at a subsequent time to FIG. 2.

FIG. 4 illustrates an overview of an example competitive binding assay detailing molecule interaction at a subsequent time to FIG. 3.

FIG. 5 illustrates a potential experiment design for a long term competitive binding assay in which the percent free labeled partner is measured and utilizing positive and negative controls in the experiment.

FIG. 6 illustrates a potential experiment design for a long term competitive binding assay in which the percent complexed labeled partner is measured and utilizing positive and negative controls in the experiment.

FIG. 7 illustrates data from a competitive binding assay involving positive and negative controls before the positive and negative controls have been utilized to scale the results from the assay.

FIG. 8 illustrates unscaled data from a long term competitive binding assay.

FIG. 9 provides data and a graph after the positive and negative controls have been used to scale the data of an experiment.

FIG. 10 provides a sample preparation schematic for an experiment designed to measure the limited partner-labeled partner complex in a competitive binding assay.

FIG. 11 provides a sample preparation schematic when measuring free labeled partner.

### DETAILED DESCRIPTION OF THE FIGURES

While the presently disclosed inventive concept(s) is susceptible of various modifications and alternative constructions, certain illustrated embodiments thereof have been shown in the drawings and will be described below in detail. It should be understood, however, that there is no intention to limit the inventive concept(s) to the specific form disclosed, but, on the contrary, the presently disclosed and claimed inventive concept(s) is to cover all modifications, alternative constructions, and equivalents falling within the spirit and scope of the inventive concept(s) as defined in the claims.

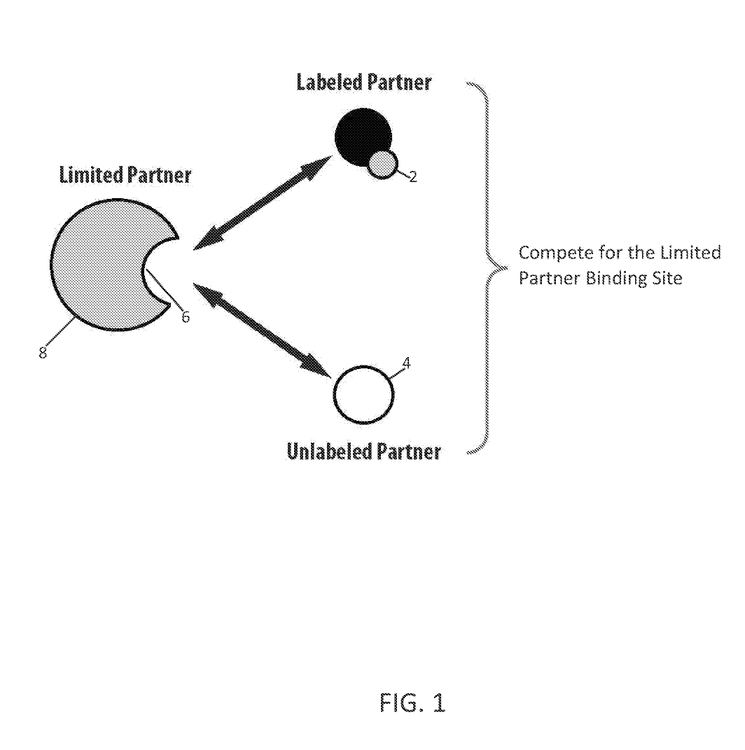

FIG. 1 illustrates conceptualization of a competitive binding assay as previously known. In a competitive binding assay, a limited partner is provided as well as a labeled partner and an unlabeled partner that bind competitively with the limited partner. The labeled partner 2 generally is discernible from the unlabeled partner. There are multiple potential aspects of the limited partner and the labeled partner that can render the labeled partner discernible from the unlabeled partner. For example, the labeled partner and unlabeled partner can be different molecules, and/or the labeled partner and unlabeled partner may have a tag or similar feature allowing a researcher to distinguish the two. The labeled partner 2 and unlabeled partner 4 competitively bind to the limited partner 8. The competitive binding can be either at the same location, for example two molecules competitively binding at the same active site on an enzyme, as depicted in FIG. 1 at a single location 6 on the limited partner, or the labeled partner and unlabeled partner can bind at different sites on the limited partner as long as the binding of one leads to the exclusion of the second. In sum, the labeled partner and unlabeled partner compete for binding with the limited partner.

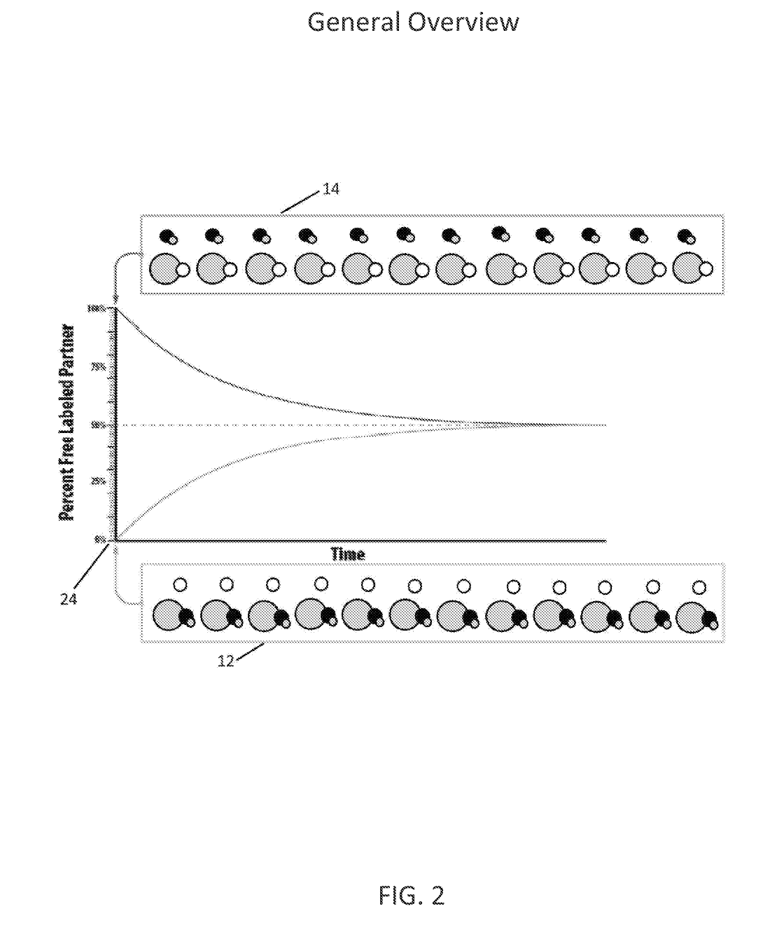

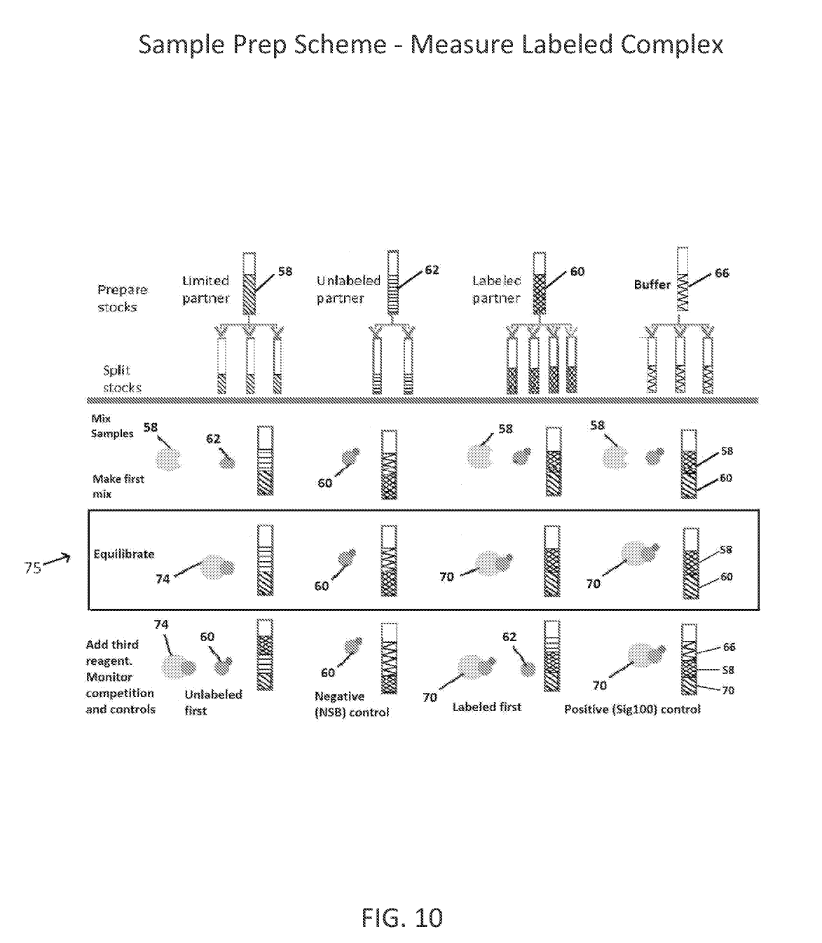

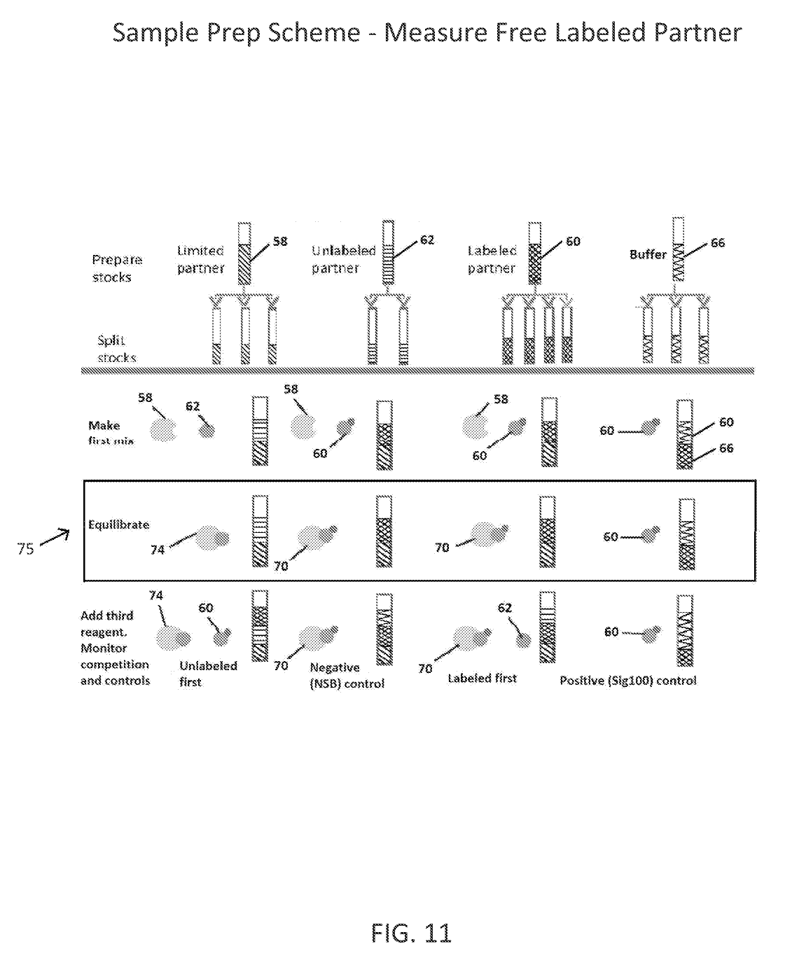

FIGS. 2-4 further illustrates conceptualization of an embodiment of a competitive binding assay in which the percent of free labeled partner in the experimental solution is measured. In FIG. 2, two starting solutions are provided and both allowed to proceed toward equilibrium, with each solution representing either the upper curve of the graph or the lower curve of the graph. In the depicted experiment the percent of free labeled partner is being measured. The lower portion 12 of the graph illustrates the reaction in which at time zero a limited partner is bound to a labeled partner and a solution of unbound unlabeled partner has been added. Time zero is defined as the furthest left point on the timeline in the graph depicted. The definition is relative to the embodiments described and the invention(s) disclosed herein are not limited to starting at a defined time but the can be varied according to the experimental design of each experiment conducted according the invention(s) disclosed herein. The upper portion 14 at time zero 24 illustrates the timing when a solution of unlabeled partner bound to the free partner has been prepared and a solution of free labeled partner has been added. In a preferred embodiment, each of the starting solutions of free labeled partner, limited partner-unlabeled partner complex, free unlabeled partner, and labeled partner-limited partner complex are prepared from stock solutions as depicted in FIGS. 10 and 11.

At time zero, the data illustrate that approximately 100% (or all) of the labeled partner is free as the free labeled partner has just been added to the solution of complexed unlabeled partner-limited partner. The starting percentages can be varied dependent on study design and it is generally thought that a similar curve will be obtained in the results. As the reaction proceeds and the time moves to the right on the graph, on the upper curve the limited partner binding with the labeled partner and unlabeled partner goes toward equilibrium, thus the percent free of labeled partner decreases. In contrast, on the lower curve the percent of free labeled partner increase as the reaction proceeds as the limited partner-labeled partner complex dissociates and the limited partner then binds with free unlabeled partner.

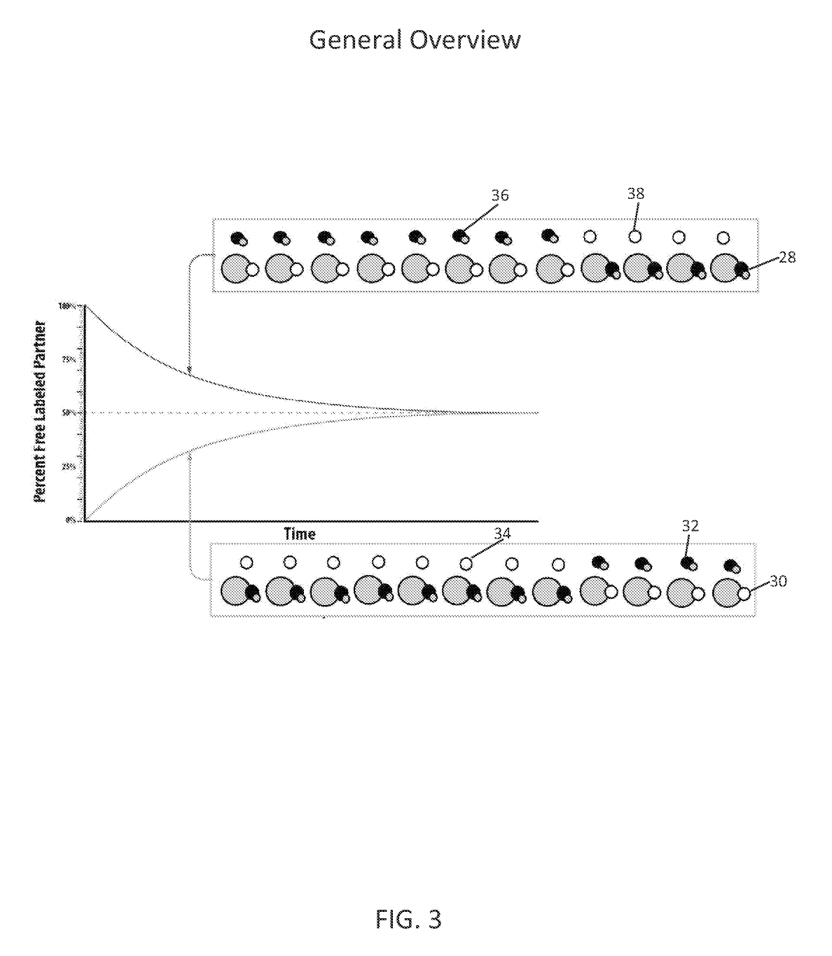

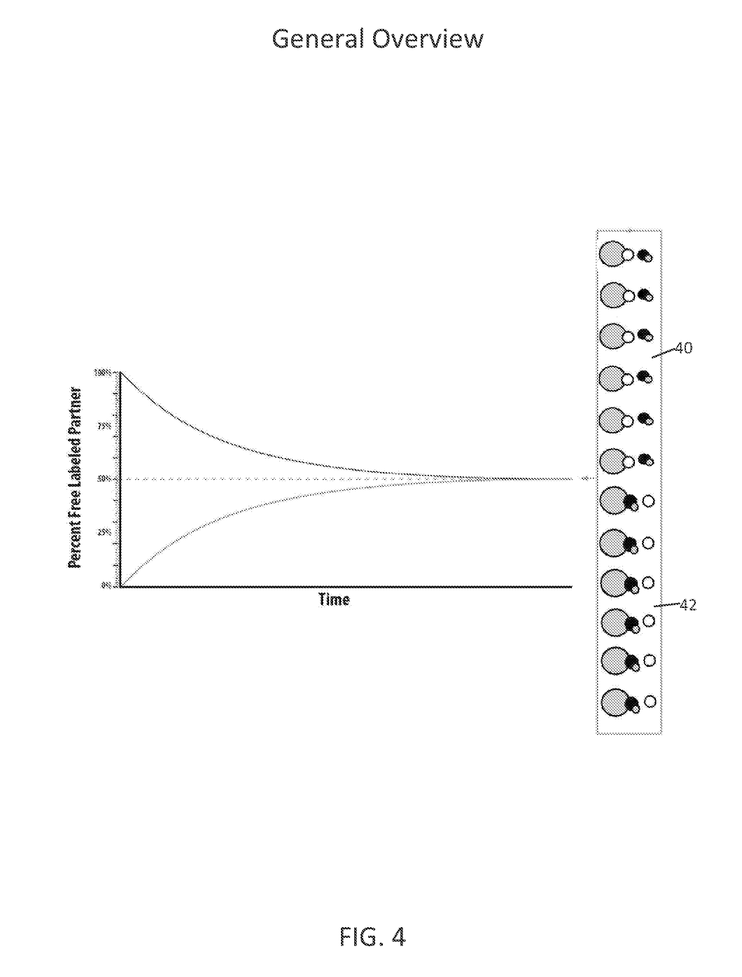

FIGS. 3 and 4 provide representation of molecule binding at time points subsequent to the time of the experiment depicted in FIG. 2. At the time point of FIG. 3, the experiment is proceeding towards equilibrium and some of the labeled partner 36 on the upper portion 28 of the graph has bound to limited partner that has dissociated from the initial limited partner-unlabeled partner complex. The converse is true on the lower curve. At the given time point, an increase percentage of the unbound labeled partner thus providing for a measurement that is proceeding upward from approximately zero in FIG. 2. There is still a greater percentage of the unlabeled partner 34 that is unbound than the labeled partner 32 that is unbound. This is depicted graphically in box 30.

FIG. 4 illustrates in a time close to or at equilibrium of the reaction. At this time point, each of the solution has approximately 50% bound and 50% free of labeled partner and unlabeled partner. This is what is thought to appear when in the depicted embodiment the reaction has completed. Each complex continues to dissociate and form new complexes, but the percent of free labeled partner and free unlabeled partner have equilibrated, thus a free limited partner has approximately an equal opportunity to bind with either labeled partner or unlabeled partner, which in each independent experiment is dependent on the association rate constant, kₒₙ, for each of the labeled partner and unlabeled partner for binding with the limited partner.

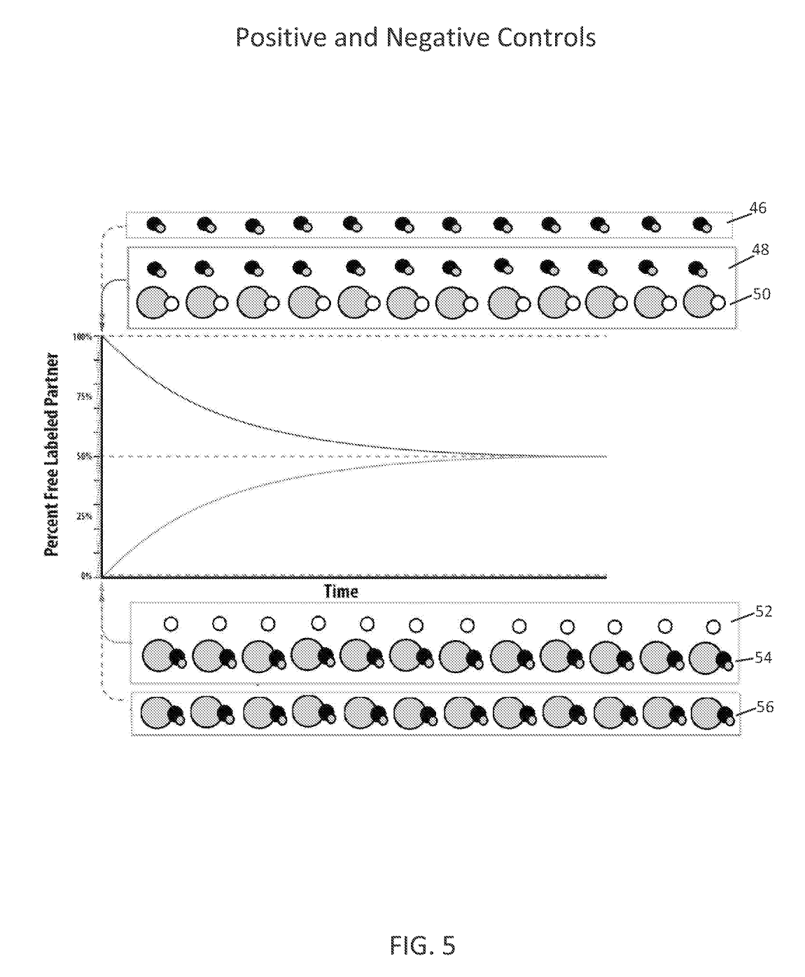

FIG. 5 depicts a preferred embodiment of the invention in which the competitive binding analysis incorporates positive and negative controls and the percent of free labeled partner is being measured for. The positive and negative controls compensate for, for example, drift in the system and/or decay in materials. The positive control 46 is a solution of free labeled partner 46 to use to scale the measurement of free labeled partner obtained from the system. In the depicted embodiment, the reaction proceeds when a second solution of free labeled partner 48 is added to a solution of complexed unlabeled partner-limited partner 50 and the solution allowed to proceed toward equilibrium (and thus yielding the results depicted in the curve as the time along the x-axis proceeds to the right). The measurements yielded by the limited partner dissociating with the unlabeled partner and associating with the labeled partner (and thus decreasing the measurement of percent free labeled partner) are scaled by the positive control of the measurement of the 100% free labeled partner 46. Thus any decay in the free labeled partner signal or change in the instrumentation is corrected for utilizing the change in signal obtained from the solution of 100% free labeled partner and corrected for by scaling for signal NSB generated from the control of the complexed labeled partner-limited partner control. Conversely, on the lower curve the negative control 56 is the labeled partner complexed with the limited partner 56. The reaction depicted by the lower curve begins with the free unlabeled partner 52 added to the complexed labeled partner-limited partner 54. This reaction is then allowed to proceed toward equilibrium, and the results scaled by the controls.

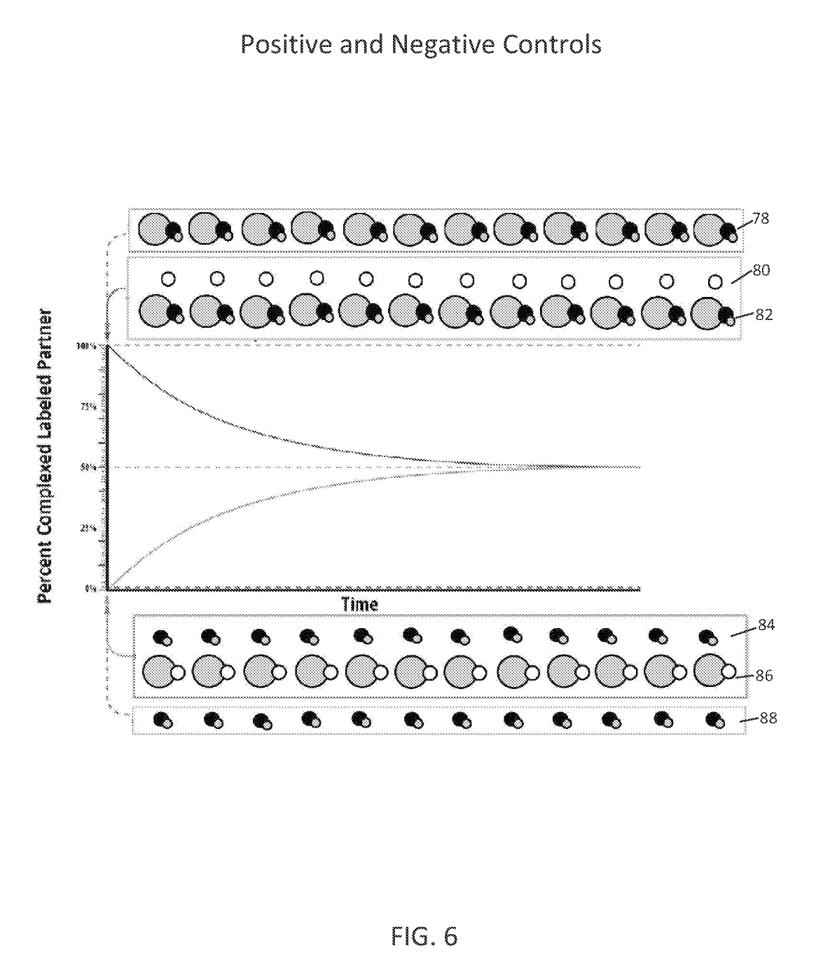

FIG. 6 depicts a preferred embodiment of the invention in which the competitive binding analysis incorporates positive and negative controls and the percent of labeled partner-limited partner is being measured. The positive control 78 is a solution of complexed labeled partner-limited partner 78 to use to scale the measurement of labeled partner-limited partner complex obtained from the system. In the depicted embodiment, the reaction proceeds when a second solution of free unlabeled partner 80 is added to a solution of complexed labeled partner-limited partner 82 and the solution allowed to proceed toward equilibrium (and thus yielding the results depicted in the curve as the time along the x-axis proceeds to the right). The measurements yielded by the limited partner dissociating with the labeled partner and associating with the unlabeled partner (and thus decreasing the measurement of percent labeled partner-limited partner complex) are scaled by the positive control of the measurement of the 100% complexed labeled partner-limited partner 78. Thus any decay in the complexed labeled partner-limited partner signal or change in the instrumentation is corrected for utilizing the change in signal obtained from the solution of 100% complexed labeled partner-limited partner and corrected for by scaling for signal NSB generated from the control solution of the free labeled partner 88 (also called the negative control). The lower curve depicts the negative control 88 as a solution of free limited partner 88. The reaction depicted by the lower curve begins with the free labeled partner 84 added to the complexed unlabeled partner-limited partner 86. This reaction is then allowed to proceed toward equilibrium, and the results scaled by the controls. In a preferred embodiment, each of the starting solutions of free labeled partner, limited partner-unlabeled partner complex, free unlabeled partner, and labeled partner-limited partner complex are prepared from stock solutions as depicted in FIGS. 10 and 11.

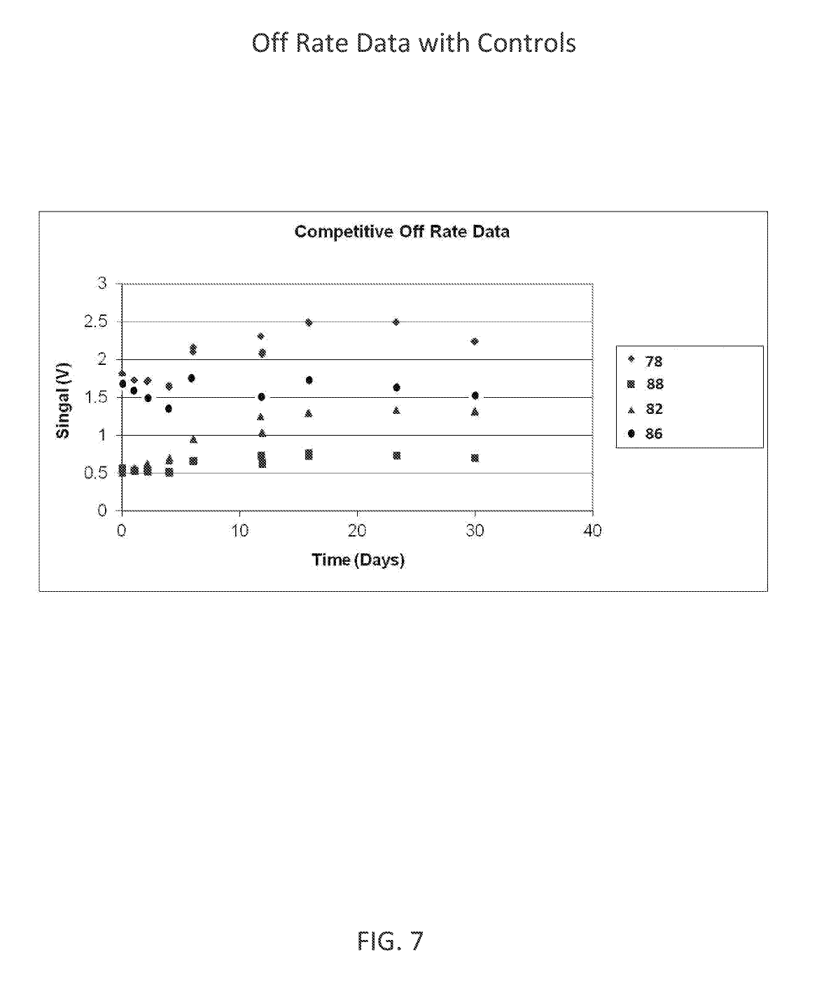

FIG. 7 illustrates the unscaled data obtained from an experiment similar to FIG. 6 including the raw data of the positive control, negative control, and competitive binding equilibrium data before the controls have been applied to the equilibrium progression results. As shown, the data before scaling with the control measurements do not provide information that can be utilized to determine a precise measurement of the kinetic rate constant (the off rate denoted as k_{off}) describing the dissociation of a non-covalent complex such as an antibody antigen complex, receptor ligand complex, etc. from the graph. The data depict the difficulty in utilizing the previous methodology known in the art.

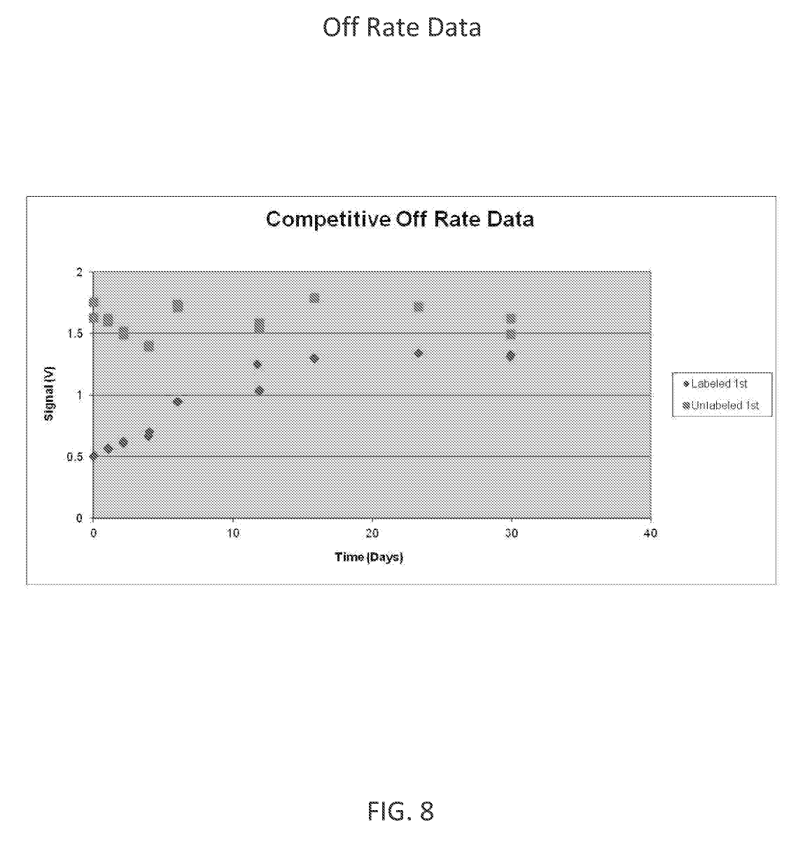

FIG. 8 illustrates competitive binding data that is typically produced over 30 to 40 day time period. A difficulty encountered by a researcher in using a competitive binding analysis is that many of the uses of the competitive binding analysis utilize a long term equilibrium. These include such things as pharmaceutical drugs, and the equilibrium rate can be very important.

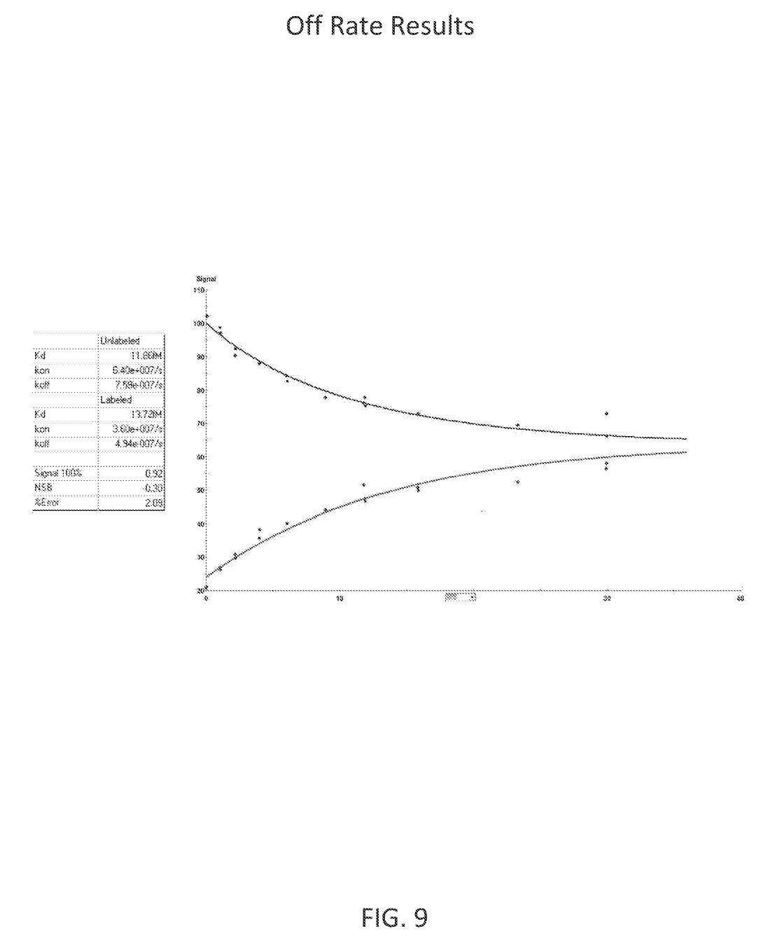

FIG. 9 illustrates the data depicted in a competitive binding experiment similar to that depicted in FIG. 7 after the data points have been scaled using the controls. The percent free of the labeled partner now are scaled according to controls. The data points now give results that are comprehensible and the k_{d} can be obtained based on the scaled data.

FIGS. 10 and 11 further illustrate sample preparation schematics that are utilized to further increase the ability of the experiment to proceed for long duration. The schematic of FIG. 10 is thought to be a preferred embodiment of preparing samples when the limited partner-labeled partner complex is being measured (as depicted in FIG. 6). The sample preparation scheme begins with preparing stocks of materials to be used in the experiment. This includes preparing stocks of the limited partner 58, unlabeled partner 62, labeled partner 60, and buffer 66. Subsequently, each of the solutions that are desired is prepared and allowed to equilibrate as depicted in box 75. This includes preparing a solution of a complexed unlabeled partner and limited partner 58, a labeled partner solution 60, a limited partner solution 58 and a complex partner labeled partner solution as a control (each of the complexed solutions is prepared by combining two separate stocks and allowing the complexed solution to equilibrate 75). In this instance, the positive control is the labeled partner-limited partner complex 70. Once the solutions equilibrate, what is left is a solution having a limited partner-unlabeled partner 74, a labeled partner solution 60 with buffer, a complexed limited partner-labeled partner solution 70 and a control of a complexed limited partner-labeled partner solution 70. Subsequently, the reagents are added to the solution to be allowed to proceed to equilibrium. This involves adding the labeled partner solution to the complexed unlabeled partner-limited partner and adding the complexed labeled partner-limited partner to the unlabeled partner. These experimental solutions are then allowed to proceed to equilibrium and the measurements are scaled utilizing the negative control having the labeled partner 60 and the positive control having the limited partner-labeled partner complex 70.

FIG. 11 depicts a similar sample schematic to FIG. 10 but with free labeled partner being measured by the hypothetical researcher (such as depicted in FIG. 2). The positive and negative controls are altered in conjunction with the free labeled partner being measured thus the positive control constitutes free labeled partner 60 whereas the negative control constitutes complexed labeled partner-limited partner. Each of the controls includes buffer to increase the total solution volume to be equivalent to the test solutions.

While certain exemplary embodiments are shown in the figures and described in this disclosure, it is to be distinctly understood that the presently disclosed inventive concept(s) is not limited thereto but may be variously embodied to practice within the scope of the following claims. From the foregoing description, it will be apparent that various changes may be made without departing from the spirit and scope of the disclosure as defined by the following claims.

1. In a competitive binding assay for determining the dissociation rate constant between two molecules, molecule A and molecule B, wherein said molecule A and said molecule B reversibly bind to form complex AB, wherein a third molecule C which competes with molecule B for binding to molecule A is also provided, wherein said molecule B and said molecule C are distinguishable from one another, and wherein said competitive binding assay comprising the following steps:
the step of providing a first solution of AB complex and providing a second solution of molecule C;
the step of providing at least one control solution, wherein said control solution is selected to establish a baseline or nsb measurement;
the step of mixing said first solution and said second solution to form a first mixture,
the step of measuring the concentration of one or more of complex AB, complex AC; molecule B, or molecule C in said first mixture and said control solution at time intervals following said step of mixing said first solution and said second solution and continuing until said competitive binding assay has sufficiently progressed to allow determination of the dissociation rate constant of complex AB; and
the step of scaling said measurement of said concentration of complex AB, complex AC, molecule B, or molecule C according to the baseline measurement obtained from said control solution.

2. The competitive binding assay of claim 1, wherein said competitive binding assay comprises the step of providing a second control solution, wherein said second control solution is configured to provide a second baseline or nsb measurement, wherein said step of scaling said measurement of said concentration of complex AB, complex AC, molecule B, or molecule C according to said first and said second control solutions.

3. The competitive binding assay of claim 1, wherein said step of providing said first solution and said second solution comprises providing a third solution comprising complex AC and a forth solution comprising molecule B, wherein said step of mixing said solutions further comprises mixing said third solution and said forth solution to form a second mixture, wherein said step of measuring the concentration of one or more of complex AB, complex AC, molecule B, or molecule C comprises measuring in both mixtures and scaling said concentration according to said baseline measurement obtained from said at least one control solution.

4. The competitive binding assay of claim 1, wherein said competitive binding assay further comprises the step of preparing a solution of molecule A, a solution of molecule B, and a solution of molecule C, splitting each of said solutions into smaller volume solutions of molecule A, molecule B, and molecule C, and combining said smaller volume solutions of molecule A and molecule B and molecule C to form said first solution of AB complex and a solution of AC complex, wherein said method further comprises the step of preparing said separate solution mixtures of AB+C and/or AC+B from said first solution of AB complex prepared from said smaller volume solutions and said AC solution from said combined smaller volume solutions, and said C and said B from said smaller volume solutions, and wherein said at least one control solutions are prepared from said small volume solutions.

5. The competitive binding assay of claim 2, wherein said competitive binding assay further comprises the step of preparing a solution of molecule A, a solution of molecule B, and a solution of molecule C, splitting each of said solutions into smaller volume solutions of molecule A, molecule B, and molecule C, and combining said smaller volume solutions of molecule A and molecule B to form said first solution of AB complex and a solution of AC complex, wherein said method further comprises the step of preparing said separate solution mixtures of AB+C and/or AC+B from said first solution of AB complex prepared from said smaller volume solutions and said AC solution from said combined smaller volume solutions, and said C and said B from said smaller volume solutions, and wherein said control solutions are prepared from said small volume solutions.
